(19) United States
(12) Patent Application Publication
(10) Pub. No.: US 2015/0140659 A1
(43) Pub. Date: May 21, 2015

(54) THREE-DIMENSIONAL CELL CULTURE SYSTEM AND MANUFACTURING METHOD THEREOF

(71) Applicant: NATIONAL TAIWAN UNIVERSITY, Taipei (TW)

(72) Inventors: PAI-CHI LI, TAIPEI (TW); PO-LING KUO, TAIPEI (TW); CHIN-HSIUNG TSAI, TAIPEI (TW)

(73) Assignee: NATIONAL TAIWAN UNIVERSITY, Taipei (TW)

(21) Appl. No.: 14/208,006

(22) Filed: Mar. 13, 2014

(30) Foreign Application Priority Data
Nov. 19, 2013 (TW) 102142071

(51) Int. Cl. C12N 5/00; G01N 33/50

(52) U.S. Cl. CPC C12N 5/0068; G01N 33/5026; C12N 2513/00; C12N 2503/02; C12N 2533/40; C12N 2535/00
USPC 435/396; 435/287.9

(57) ABSTRACT

A three-dimensional cell culture system for an imaging system to observe a cell image includes at least two cell culture layers formed by a solution having a photo-polymerizable monomer, a bio-molecule, an acoustic scattering medium solution and a cell culture medium. After placing a cell into the two cell culture layers, the two cell culture layers are laminated to form a three-dimensional culture laminating layer for culturing the cell. After forming the three-dimensional culture laminating layer, at least one cell-locating layer having a polyethylene glycol diacrylate (PEGDA) solution, the acoustic scattering medium solution, a plurality of photoacoustic markers and the cell culture medium is positioned into the three-dimensional culture laminating layer so as to form the three-dimensional cell culture system. The imaging system is constructed according to a theory selected from one of optics, acoustics, optoacoutics and acousto-optics.

This application claims the benefit of Taiwan Patent Application Serial No. 102142071, filed Nov. 19, 2013, the subject matter of which is incorporated herein by reference.

### BACKGROUND OF INVENTION

1. Field of the Invention

The invention relates to a three-dimensional cell culture system and a manufacturing method of the three-dimensional cell culture system, and more particularly to the three-dimensional cell culture system and the corresponding manufacturing method that the cell to be tested can be monitored by an imaging system formed according to the optics, acoustics, optoacoutics, or acousto-optics.

2. Description of the Prior Art

Recently, remarkable progress in various bio-medical research, especially in drug development and pathology, has been made. In these researches, cell culturing, mainly two-dimensional or three-dimensional cell culture, is one of the crucial and basic steps in bio-medical experiments.

In the art, the two-dimensional cell culture has been widely applied to various types of cells. However, from observations upon the cell behavior or the published results in those two-dimensional research, it is noticed that various human physiological environments may not be applied. A well-known fact is that the cell culture under a three-dimensional environment might express different results to that under a two-dimensional culture. For example, majority of the cell cultures under the two-dimensional environment can't develop into an concrete organizing form similar to those obtained under the three-dimensional culture environment or in real human tissue. Namely, it is the research result obtained from the three-dimensional cell culture that can be really applicable to the coming-up biomedical application.

Particularly, the spatially dynamic research in the extracellular matrix (ECM) can perform in-depth observation and analysis upon the group activity of the adhered cells or the signal transmission among cells. Such kind of research can provide plenty and significant information to the life science and/or clinical research. The spatial dynamics of ECM is characterized in its local and short-term elastic deformation. In addition, the long-term structure remodeling of ECM might change the mechanical properties of the culture substrate.

Currently, most of the aforesaid dynamic research use the optic imaging technique to proceed the monitoring. However, light scattering and bleaching introduce unexpected limitations for the foregoing monitoring. Therefore, techniques tracking the 3D characteristics of ECM deformation with a large spatial range within considerably short time scales are of a great desire.

### SUMMARY OF THE INVENTION

In view of the published documents regarding the three-dimensional cell culture, the problem of light scattering and bleaching in the traditional optics imaging art does degrade the observation in these research. Accordingly, it is the primary object of the present invention to provide a three-dimensional cell culture system and a manufacturing method of the three-dimensional cell culture system to include a polymer solution with optoacoutic or acousto-optic substances, such that the optic, acoustic, optoacoutic and/or acousto-optic imaging system can observe easily the cells to be tested.

In the present invention, the three-dimensional cell culture system applies an imaging system to observe at least one cell image of cells to be tested, and comprises at least two cell culture layers and at least one cell-locating layer. The cell culture layer is formed by a polymer solution having at least one monomer capable of photo-polymerization, a first polymerized solution formed by polymerizing bio-molecules capable of providing cell recognition and providing signals to one of the cells to be tested, a second polymerized solution formed by polymerizing an acrylate, a polyethylene glycol and a cell adhesive peptide (CAP), an acoustic scattering medium solution and a cell culture medium, in which the at least two cell culture layers so are laminated to form a unique three-dimensional culture laminating layer for culturing the cells to be tested.

The cell-locating layer is formed by a polyethylene glycol diacrylate (PEGDA) solution, the acoustic scattering medium solution, a plurality of photoacoustic markers and the cell culture medium, in which the cell-locating layer has a preset shape and a preset position for providing the imaging system to perform positioning and is located in the three-dimensional culture laminating layer. The imaging system is constructed according to a theory selected from one of optics, acoustics, optoacoutics and acousto-optics, and the at least one cell image is one of an ultrasonic image, a photoacoustic image, an elasticity image, a molecular image and a distribution image.

Coherently, in the present invention, the manufacturing method of the aforesaid three-dimensional cell culture system is to produce the three-dimensional cell culture system for the imaging system to observe at least one cell image of the cells to be tested. The manufacturing method comprises the steps of: (a) preparing a polymer solution having at least one monomer capable of photo-polymerization, a first polymerized solution formed by polymerizing bio-molecules capable of providing cell recognition and providing signals to one of the cells, a second polymerized solution formed by polymerizing an acrylate, a polyethylene glycol and a cell adhesive peptide (CAP), an acoustic scattering medium solution and a cell culture medium; (b) applying the polymer solution to prepare at least two cell culture layers; (c) placing one of the cells to be tested on the at least two cell culture layers; (d) laminating the at least two cell culture layers so as to form a three-dimensional culture laminating layer having the one of the cells to be tested cultured in the three-dimensional culture laminating layer; (e) preparing at least one cell-locating layer having a polyethylene glycol diacrylate (PEGDA) solution, the acoustic scattering medium solution, a plurality of photoacoustic markers and the cell culture medium; and (f) positioning the at least one cell-locating layer in the three-dimensional culture laminating layer so as to form the three-dimensional cell culture system, wherein the at least one cell-locating layer has a preset shape and a preset position. Again, the imaging system is constructed according to a theory selected from one of optics, acoustics, optoacoutics and acousto-optics, and the at least one cell image is one of an ultrasonic image, a photoacoustic image, an elasticity image, a molecular image and a distribution image.

In another aspect of the manufacturing method in accordance with the present invention, the manufacturing method of the three-dimensional cell culture system is also to produce the three-dimensional cell culture system for the imaging system to observe at least one cell image of the cells to be tested, and comprises the steps of: (a) preparing a polymer solution having at least one monomer capable of photo-polymerization, a first polymerized solution formed by polymerizing bio-molecules capable of providing cell recognition and providing signals to one of the cells, a second polymerized solution formed by polymerizing an acrylate, a polyethylene glycol and a cell adhesive peptide (CAP), an acoustic scattering medium solution and a cell culture medium; (b) applying the polymer solution to prepare at least two cell culture layers; (c) laminating the at least two cell culture layers so as to form a three-dimensional culture laminating layer; (d) preparing at least one cell-locating layer having a polyethylene glycol diacrylate (PEGDA) solution, the acoustic scattering medium solution, a plurality of photoacoustic markers and the cell culture medium; (e) positioning the at least one cell-locating layer in the three-dimensional culture laminating layer so as to form the three-dimensional cell culture system, wherein the at least one cell-locating layer has a preset shape and a preset position; and (f) placing one of the cells to be tested in the three-dimensional culture laminating layer of the three-dimensional cell culture system. Similarly, the imaging system is constructed according to a theory selected from one of optics, acoustics, optoacoutics and acousto-optics, and the at least one cell image is one of an ultrasonic image, a photoacoustic image, an elasticity image, a molecular image and a distribution image.

In the present invention, the monomer is formed by an acrylate and a polyethylene glycol (PEG), and the bio-molecule is one of an enzyme sensitive peptide (ESP), a growth factor and a chemokine; in which the enzyme sensitive peptide is formed by amino acids, and the acoustic scattering medium solution is a SiO₂ suspension.

In the present invention, the first polymerized solution has a percent weight in volume ranged from 3-5%, the second polymerized solution has a percent weight in volume ranged from 1-1.5%, the acoustic scattering medium solution has a percent weight in volume ranged from 0.1-0.3%, and the cell culture medium is formed by a Dulbecco's modified eagle medium, a fetal bovine serum (FBS) and antibiotics, and the polyethylene glycol diacrylate solution of the cell-locating layer has a percent weight in volume ranged from 3-5%.

In the present invention, the polymer solution and the cell-locating layer further include an ultraviolet-initiator having a percent weight in volume ranged from 0.1-1%, and the photoacoustic marker is one of a nanogold rod, a dye and a graphite; wherein, in the case that the photoacoustic marker is the nanogold rod, a concentration of the nanogold rod in the cell-locating layer is larger than 5*10⁹ unit/ml. Further, the cells to be tested are cells sampled or derived from one of a human organ and an animal and have a volume concentration ranged from 10⁴-10⁶ cell/ml, and wherein the imaging system is one of an ultrasonic system, an optoacoutic imaging system, an elastic imaging system and an optic imaging system.

By providing the photo-polymerizable monomer and bio-molecules, the polyethylene glycol diacrylate solution and the photoacoustic markers to the three-dimensional cell culture system and the manufacturing method of the three-dimensional cell culture system in accordance with the present invention, the three-dimensional cell culture system can thus apply the imaging system to observe the photoacoustic image easily, and thereby the prior-art problem of the three-dimensional culture substrate unable to be observed due to lack of photoacoustic markers for photoacoustic imaging is then resolved.

In addition, for the acoustic scattering medium solution is added into the three-dimensional cell culture system, the ultrasonic multi-wave imaging technique can then be integrated to observe the structural change or cell distribution in the three-dimensional cell culture. Further, the overall physical variation and the cellular dynamics between the cells to be tested and the environment can be measured, and also various cell images can be obtained. Upon such an arrangement, the observation upon the three-dimensional cell culture can then be versatile.

All these objects are achieved by the three-dimensional cell culture system and the corresponding manufacturing method of the three-dimensional cell culture system described below.

### BRIEF DESCRIPTION OF THE DRAWINGS

The present invention will now be specified with reference to its preferred embodiment illustrated in the drawings, in which:

FIG. 1 is a schematic perspective view of a preferred embodiment of the three-dimensional cell culture system in accordance with the present invention;

FIG. 2 is a schematic view showing the experiment setup for an imaging system to observe the three-dimensional cell culture system of FIG. 1;

FIG. 3 is a flowchart of a preferred embodiment of the manufacturing method of a three-dimensional cell culture system in accordance with the present invention;

FIG. 4 is a schematic view of the cell culture layer of FIG. 1;

FIG. 5 shows an experiment arrangement for cell culturing in accordance with the present invention;

FIG. 6A through FIG. 6C demonstrate different stages of cell culturing by an ultrasonic imaging system in accordance with the present invention;

FIG. 7A demonstrates cell culturing of a second embodiment of the three-dimensional cell culture system in accordance to with the present invention;

FIG. 7B demonstrates cell culturing of a third embodiment of the three-dimensional cell culture system in accordance with the present invention;

FIG. 8A is a schematic side view of FIG. 7A;

FIG. 8B is a schematic side view of FIG. 7B; and

FIG. 9A through FIG. 9C show images of cell culturing in accordance with the present invention.

### DESCRIPTION OF THE PREFERRED EMBODIMENT

The invention disclosed herein is directed to a three-dimensional cell culture system and a manufacturing method of the three-dimensional cell culture. In the following description, numerous details are set forth in order to provide a thorough understanding of the present invention. It will be appreciated by one skilled in the art that variations of these specific details are possible while still achieving the results of the present invention. In other instance, well-known components are not described in detail in order not to unnecessarily obscure the present invention.

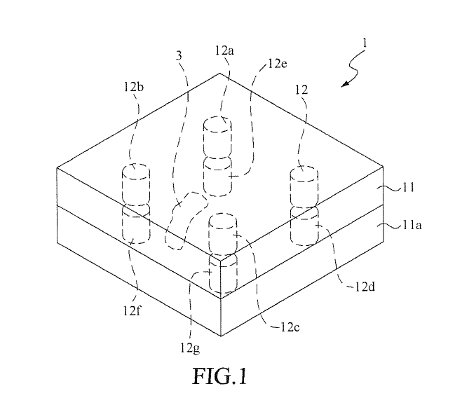

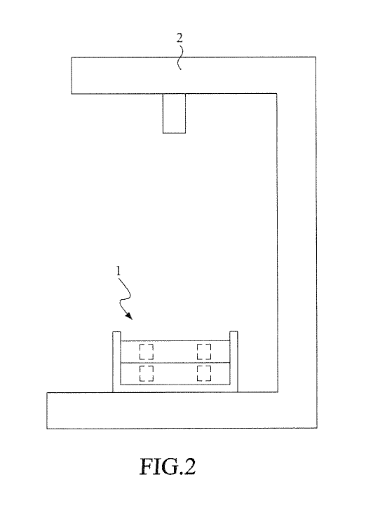

Refer now to FIG. 1 and FIG. 2, in which FIG. 1 is a schematic perspective view of a preferred embodiment of the three-dimensional cell culture system in accordance with the present invention, and FIG. 2 is a schematic view showing the experiment setup for an imaging system to observe the three-dimensional cell culture system of FIG. 1. As shown, the three-dimensional cell culture system 1 of the present invention applies an imaging system 2 to observe at least one cell image (not shown in the figures) of cells to be tested 3. The three-dimensional cell culture system 1 comprises at least two cell culture layers 11, 11a and a plurality of cell-locating layers 12, 12a, 12b, 12c, 12d, 12e, 12f and 12g.

The imaging system 2 is constructed according to a theory selected from one of optics, acoustics, optoacoutics and acousto-optics. Namely, the imaging system 2 is at least one or a combination of an ultrasonic system, an optoacoutic imaging system, an elastic imaging system and an optic imaging system. Also, the cells to be tested 3 are either cells of a human organ or animal cells.

In this preferred embodiment, each of the cell culture layers 11 and 11a is formed by a polymer solution having a first polymerized solution, a second polymerized solution, an acoustic scattering medium solution, a cell culture medium and an ultraviolet-initiator. The first polymerized solution is formed by polymerizing at least one monomer capable of photo-polymerization and bio-molecules capable of providing cell recognition and providing signals to one of the cells to be tested. In the preferred embodiment, the photo-polymerizable monomer is formed by an acrylate and a polyethylene glycol, in which the molecular weight of the polyethylene glycol is 3500 Da (Dalton). In other embodiment of the present invention, any monomer or polymer capable of photo-polymerization can also be an alternative of the foregoing photo-polymerizable substance.

In the present invention, the bio-molecule is one of an enzyme sensitive peptide (ESP), a growth factor and a chemokine. In this embodiment, the bio-molecule is the enzyme sensitive peptide made up of amino acids. The enzyme sensitive peptide can be recognized and digested by matrixmetalloproteinase-1 (MMP-1) from cells. The sequence of peptide can be adjusted according to the enzyme from the cell to be tested. Further, in this preferred embodiment, the first polymerized solution has a percent weight in volume ranged from 3-5%.

The second polymerized solution is formed by polymerizing an acrylate, a polyethylene glycol and a cell adhesive peptide (CAP); in which the polyethylene glycol has a molecular weight of 3500 Da (Dalton). The second polymerized solution is to promote the cells to attach to the surrounding object and can be adjusted to different peptides according to types of the cells. In addition, the second polymerized solution has a percent weight in volume ranged from 1-1.5%.

The acoustic scattering medium solution is a SiO₂ suspension, in which the SiO₂ particles have a diameter ranged from 1-5 μm. In addition, the acoustic scattering medium solution has a percent weight in volume ranged from 0.1-0.3%.

In the preferred embodiment, the cell culture medium is formed by a Dulbecco's modified eagle medium, 10% of the fetal bovine serum (FBS) and 1% of the antibiotics.

The ultraviolet-initiator has a percent weight in volume ranged from 0.1-1%. Preferably, the ultraviolet-initiator is the Irgacure 2959. In the present invention, the reason of adding the ultraviolet-initiator into the first polymerized solution is because the first polymerized solution of the preferred embodiment is formed under a controlled environment of 365 nm UV light, 10-30 seconds reaction time, and room temperature. Hence, an ultraviolet-initiator is needed to act as a photocatalyst for the 365 nm UV light.

As shown, the two cell culture layers 11 and 11a are laminated to form a unique three-dimensional culture laminating layer for culturing the cells 3 to be tested. Preferably, the cells 3 to be tested in the three-dimensional culture laminating layer have a volume concentration ranged from 10⁴-10⁶ cell/ml.

In FIG. 1, each of the cell-locating layers 12, 12a, 12b, 12c, 12d, 12e, 12f and 12g is formed by a polyethylene glycol diacrylate (PEGDA) solution, the aforesaid acoustic scattering medium solution, a plurality of photoacoustic markers and a cell culture medium. The polyethylene glycol diacrylate solution has a percent weight in volume ranged from 3-5%. The photoacoustic marker is one of a nanogold rod, a dye and a graphite; in which, in the case that the photoacoustic marker is the nanogold rod, a concentration of the nanogold rod in any of the cell-locating layers 12, 12a, 12b, 12c, 12d, 12e, 12f and 12g is larger than 5*10⁹ unit/ml.

Further, each of the cell-locating layers 12, 12a, 12b, 12c, 12d, 12e, 12f and 12g in the three-dimensional culture laminating layer has a preset shape and a preset position, and is to help the imaging system 2 to perform positioning; such that cell images of the cells 3 to be tested can be precisely obtained and thus the corresponding cell growth stages can be observed as well. In accordance with the imaging system 2, the cell image can be one of an ultrasonic image, a photoacoustic image, an elasticity image, a molecular image and a distribution image. The preset shape can be a cylindrical shape or a polygon shape. Preferably, the cell-locating layers 12, 12a, 12b, 12c, 12d, 12e, 12f and 12g are all cylindrically shaped in the three-dimensional culture laminating layer.

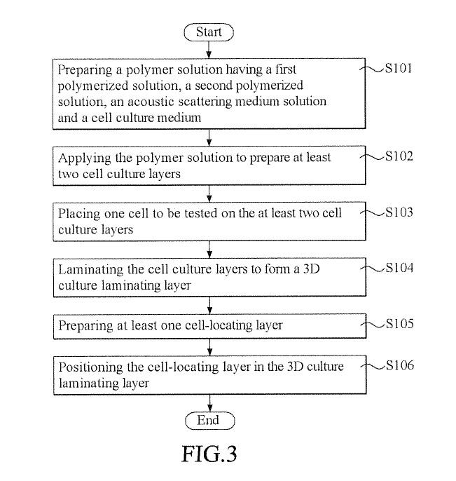

Referring now to FIG. 3, a flowchart of a preferred embodiment of the corresponding manufacturing method of the aforesaid three-dimensional cell culture system in accordance with the present invention is shown. The manufacturing method includes the steps of: (also referring to FIG. 1 and FIG. 2)

Step S101: preparing a polymer solution having a first polymerized solution formed by polymerizing at least one monomer and bio-molecules, a second polymerized solution formed by polymerizing an acrylate, a polyethylene glycol and a cell adhesive peptide (CAP), an acoustic scattering medium solution and a cell culture medium;

Step S102: applying the polymer solution to prepare at least two cell culture layers 11;

Step S103: placing one of the cells 3 to be tested on the at least two cell culture layers 11;

Step S104: laminating the at least two cell culture layers 11 so as to form a three-dimensional culture laminating layer having the one of the cells 3 to be tested positioned in the three-dimensional culture laminating layer;

Step S105: preparing at least one cell-locating layer 12, 12a, 12b, 12c, 12d, 12e, 12f or 12g having a polyethylene glycol diacrylate (PEGDA) solution, a SiO₂ suspension, a plurality of photoacoustic markers and the cell culture medium; and

Step S106: positioning the at least one cell-locating layer 12, 12a, 12b, 12c, 12d, 12e, 12f or 12g in the three-dimensional culture laminating layer so as to form the three-dimensional cell culture system 1, in which the cell-locating layer 12, 12a, 12b, 12c, 12d, 12e, 12f or 12g has a preset shape and a preset position.

The components and the corresponding percent weight in volume for individual elements in the method for producing the three-dimensional cell culture system 1 are the same as those described in the previous paragraphs for elucidating FIG. 1 and FIG. 2, and thus are omitted herein. However, in another embodiment, the Step S103 can be shifted to be performed after the three-dimensional cell culture system 1 is formed. For example, in the case that the three-dimensional cell culture system 1 for culturing the cells 3 to be tested is a porous scaffold, the cells 3 to be tested are placed into the porous three-dimensional culture laminating layer after the step of laminating the cell culture layers 11 to form the three-dimensional culture laminating layer and the step of setting up the cell-locating layers 12, 12a, 12b, 12c, 12d, 12e, 12f and 12g.

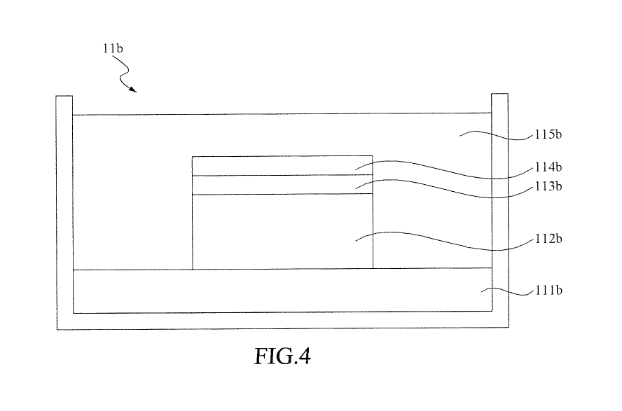

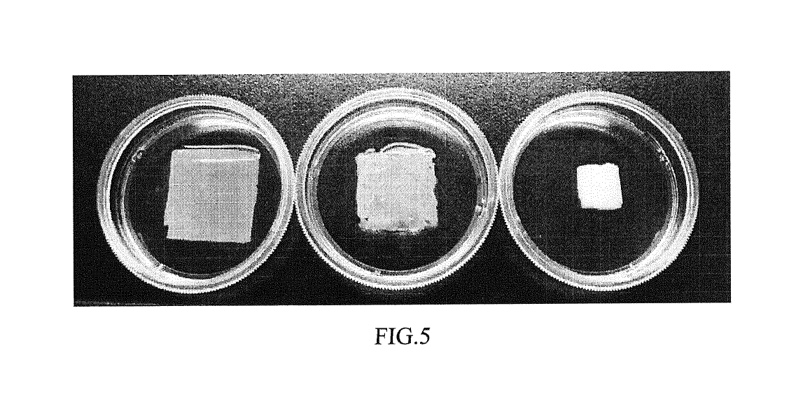

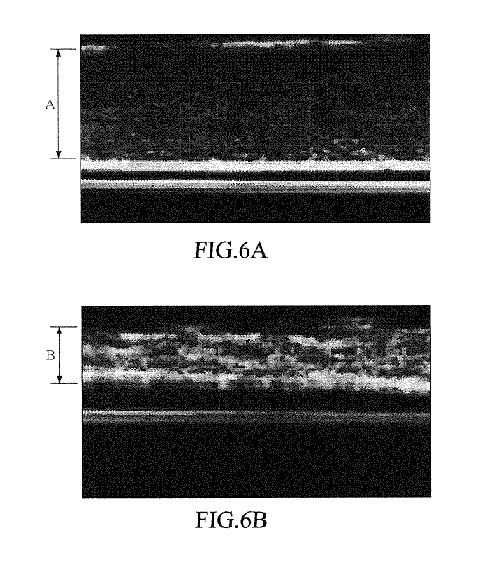

In order to have the ordinary-skill person in the art to better understand the technique of the cell culture layer of the present invention, following examples are provided. Referring now to FIG. 4 through FIG. 6C, FIG. 4 is a schematic view of the cell culture layer of FIG. 1, FIG. 5 shows an experiment arrangement for cell culturing in accordance with the present invention, and FIG. 6A through FIG. 6C demonstrate different stages of cell culturing by an ultrasonic imaging system in accordance with the present invention.

As illustrated, the cell culture layer 11b of the preferred embodiment of the present invention includes an agarose fixation layer 111b, a bulk agarose layer 112b, a co-gel layer 113b, a collagen gel layer 114b and a cell culture medium 115b. The collagen gel layer 114b contains the acoustic scattering medium solution. In the cell culture layer 11b of the three-dimensional cell culture system 1, the first polymerized solution formed by polymerizing at least one monomer and bio-molecules, the second polymerized solution formed by polymerizing an acrylate, a polyethylene glycol and a cell adhesive peptide (CAP), the acoustic scattering medium solution and the cell culture medium are contained in the agarose fixation layer 111b, the bulk agarose layer 112b, the co-gel layer 113b, the collagen gel layer 114b and the cell culture medium 115b, respectively.

Preferably, the agarose fixation layer 111b has a thickness of 1 mm, the bulk agarose layer 112b has a thickness of 7 mm, the co-gel layer 113b has a thickness of 1 mm, and the collagen gel layer 114b also has a thickness of 1 mm. However, the aforesaid thicknesses are not fixed but adjustable to meet various embodying situations.

For example, in an experiment implementing the aforesaid cell culture layer 11b, the three-dimensional culture laminating layer can be formed as the one shown in the left-hand side of FIG. 5. Also, it is noted that, during the process of forming any of FIG. 5, all the boundaries are formed without any physical constraint. The cells of lung adenocarcinoma with individual concentrations are planted into the left-hand side structure of FIG. 5. After a 5-day standing time, the culture stages are shown in the middle structure or the right-hand side structure of FIG. 5. In particular, the left-hand side structure of FIG. 5 also demonstrates the 5-day stage of the culture without planting any of the cells of lung adenocarcinoma. In the middle structure of FIG. 5, the cells of lung adenocarcinoma with a concentration of 5×10⁴ cells/ml are cultured, while, in the right-hand side structure of FIG. 5, the cells of lung adenocarcinoma with a concentration of 10⁷ cells/ml are cultured. From FIG. 5, it is directly observed that the cell culturing with a higher concentration demonstrates a significant change (shrinking in volume) in the cell culture layer.

In the case that a solid-boundary disk is used to culture the cells of lung adenocarcinoma as that in FIG. 5, then the change for the higher-concentration cell culture layer would be a smaller thickness thereof. If a B-mode of the ultrasonic system is applied to perform the observation, the corresponding culturing stages are shown in FIG. 6A through FIG. 6C. Namely, FIG. 6A shows the ultrasonic image for the cell culture without any cell of lung adenocarcinoma, FIG. 6B shows the ultrasonic image for the cell culture with the cells of lung adenocarcinoma having a concentration of 5×10⁴ cells/ml, and FIG. 6C shows the ultrasonic image for the cell culture with the cells of lung adenocarcinoma having a concentration of 10⁷ cells/ml. As shown, thickness A is larger than thickness B, but smaller in density. Also, thickness B is larger than thickness C, but smaller in density. Such a result explains that various quantity in the cells of lung adenocarcinoma do change the volume of the extracellular matrix.

In another embodiment, an observation upon the elasticity image may tell the propagation speed of the shear wave and the elastic modulus. For the example described above, by utilizing the elasticity image to observe the experiments, it can be calculated that the propagation speed of the shear wave in FIG. 6A is about 0.3 m/s and the corresponding elastic modulus is 70.62±22.84 Pa, the propagation speed of the shear wave in FIG. 6B is about 0.59 m/s and the corresponding elastic modulus is 163.76±26.7 Pa, and the propagation speed of the shear wave in FIG. 6C is about 0.77 m/s and the corresponding elastic modulus is 262.44±4.9 Pa. Therefore, in the preferred three-dimensional cell culture system 1 of the present invention, the cell images as well as the mechanical properties between the cells 3 to be tested and the cell culture layer 11b can then be successfully observed.

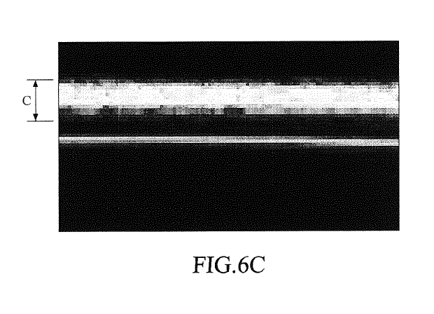

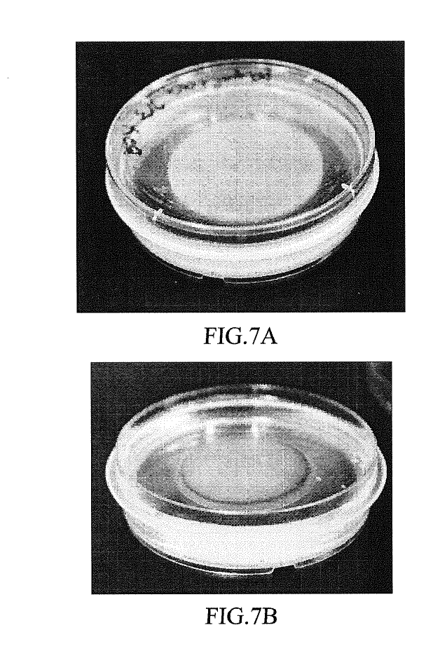

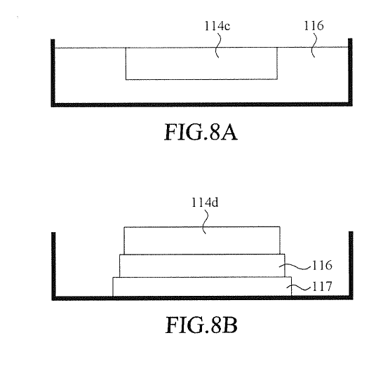

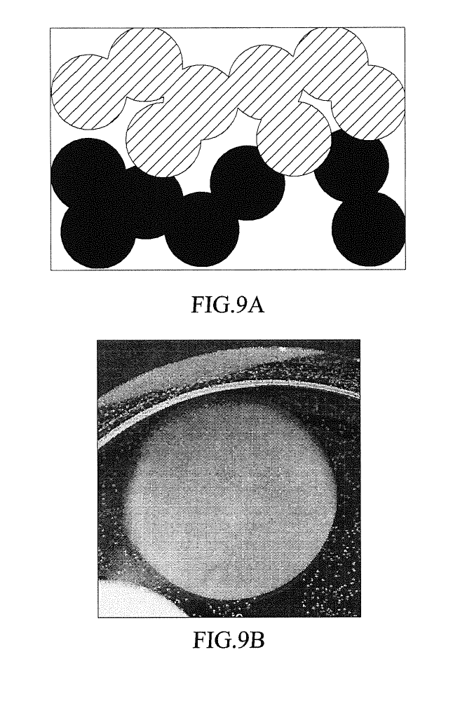

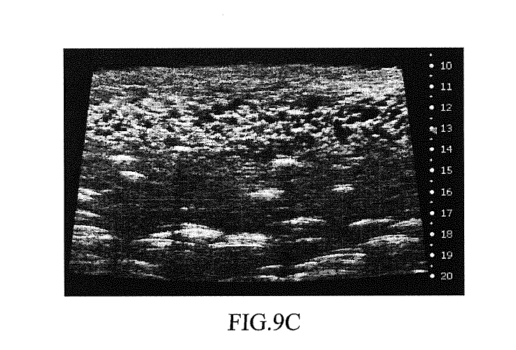

Referring now to FIG. 7A through FIG. 9C, FIG. 7A demonstrates cell culturing of a second embodiment of the three-dimensional cell culture system in accordance with the present invention, FIG. 7B demonstrates cell culturing of a third embodiment of the three-dimensional cell culture system in accordance with the present invention, FIG. 8A is a schematic side view of FIG. 7A, FIG. 8B is a schematic side view of FIG. 7B, and FIG. 9A through FIG. 9C show images of cell culturing in accordance with the present invention.

Referring to FIG. 8A (also FIG. 7A), in the cell culture layer of the second embodiment, the collagen gel layer 114c is contained in a monomer layer 116. However, in the cell culture layer of the third embodiment (referring to FIG. 7B and FIG. 8B), the collagen gel layer 114d is located above the monomer layer 116 that is laminated on a glass plate 117.

In addition, for the embodiment of the three-dimensional cell culture system formed as a porous scaffold, the cell culture layer is provided as a porous substrate having plenty cavities or holes to culture therein the cells 3 to be tested. By implementing a suitable imaging system 2, cell images can be obtained as ultrasonic images, photoacoustic images, elasticity images, molecular images or distribution images. For example, FIG. 9A shows a schematic molecular structure, FIG. 9B shows a practical camera image, and FIG. 9C shows a corresponding ultrasonic image. In this observation shown in FIG. 9C, several holes with individual diameters tiny to about 500 m are observed.

In the present invention, the inclusion of the cell-locating layer 12 makes possible the observation of the photoacoustic images, such that more experimental data regarding the growth stages of the cells to be tested 3 can be obtained.

By providing the photo-polymerizable monomer and bio-molecules, the polyethylene glycol diacrylate solution and the photoacoustic markers to the three-dimensional cell culture system and the manufacturing method of the three-dimensional cell culture system in accordance with the present invention, the three-dimensional cell culture system can thus apply the imaging system to observe the photoacoustic image easily, and thereby the prior-art problem of the three-dimensional culture substrate unable to be observed due to lack of photoacoustic markers for photoacoustic imaging is then resolved.

In addition, for the acoustic scattering medium solution is aided into the three-dimensional cell culture system, the ultrasonic multi-wave imaging technique can then be integrated to observe the structural change or cell distribution in the three-dimensional cell culture. Further, the overall physical variation and the cellular dynamics between the cells to be tested and the environment can be measured, and also various cell images can be obtained. Upon such an arrangement, the observation upon the three-dimensional cell culture can then be versatile.

While the present invention has been particularly shown and described with reference to a preferred embodiment, it will be understood by those skilled in the art that various changes in form and detail may be without departing from the spirit and scope of the present invention.

What is claimed is:

1. A three-dimensional cell culture system, applying an imaging system to observe at least one cell image of cells to be tested, comprising:
at least two cell culture layers, formed by a polymer solution having at least one monomer capable of photo-polymerization, a first polymerized solution formed by polymerizing bio-molecules capable of providing cell recognition and providing signals to one of the cells, a second polymerized solution formed by polymerizing an acrylate, a polyethylene glycol and a cell adhesive peptide (CAP), an acoustic scattering medium solution and a cell culture medium, wherein the at least two cell culture layers are laminated to form a unique three-dimensional culture laminating layer for culturing the cells to be tested; and
at least one cell-locating layer, formed by a polyethylene glycol diacrylate (PEGDA) solution, the acoustic scattering medium solution, a plurality of photoacoustic markers and the cell culture medium, wherein the at least one cell-locating layer having a preset shape and a preset position for providing the imaging system to perform positioning is located in the three-dimensional culture laminating layer;
wherein the imaging system is constructed according to a theory selected from one of optics, acoustics, optoacoutics and acousto-optics, and the at least one cell image is one of an ultrasonic image, a photoacoustic image, an elasticity image, a molecular image and a distribution image.

2. The three-dimensional cell culture system according to claim 1, wherein the monomer is formed by an acrylate and a polyethylene glycol (PEG), and the bio-molecule is one of an enzyme sensitive peptide (ESP), a growth factor and a chemokine.

3. The three-dimensional cell culture system according to claim 2, wherein the enzyme sensitive peptide is formed by amino acids.

4. The three-dimensional cell culture system according to claim 1, wherein the acoustic scattering medium solution is a SiO₂ suspension.

5. The three-dimensional cell culture system according to claim 1, wherein the first polymerized solution has a percent weight in volume ranged from 3-5%, the second polymerized solution has a percent weight in volume ranged from 1-1.5%, the acoustic scattering medium solution has a percent weight in volume ranged from 0.1-0.3%, and the cell culture medium is formed by a Dulbecco's modified eagle medium, a fetal bovine serum (FBS) and antibiotics.

6. The three-dimensional cell culture system according to claim 1, wherein the polyethylene glycol diacrylate solution of the cell-locating layer has a percent weight in volume ranged from 3-5%.

7. The three-dimensional cell culture system according to claim 1, wherein the polymer solution and the cell-locating layer further include an ultraviolet-initiator having a percent weight in volume ranged from 0.1-1%.

8. The three-dimensional cell culture system according to claim 1, wherein the photoacoustic marker is one of a nanogold rod, a dye and a graphite; wherein, in the case that the photoacoustic marker is the nanogold rod, a concentration of the nanogold rod in the cell-locating layer is larger than 5*10⁹ unit/ml.

9. The three-dimensional cell culture system according to claim 1, wherein the cells to be tested are cells sampled or derived from one of a human organ and an animal and have a volume concentration ranged from 10⁴-10⁶ cell/ml.

10. The cell-locating layer in the three-dimensional cell culture system according to claim 1, wherein the preset shape is a cylindrical shape.

11. The three-dimensional cell culture system according to claim 1, wherein the imaging system is one of an ultrasonic system, an optoacoutic imaging system, an elastic imaging system and an optic imaging system.

12. A manufacturing method of a three-dimensional cell culture system, to produce the three-dimensional cell culture system for an imaging system to observe at least one cell image of cells to be tested, comprising the steps of:
(a) preparing a polymer solution having at least one monomer capable of photo-polymerization, a first polymerized solution formed by polymerizing bio-molecules capable of providing cell recognition and providing signals to one of the cells, a second polymerized solution formed by polymerizing an acrylate, a polyethylene glycol and a cell adhesive peptide (CAP), an acoustic scattering medium solution and a cell culture medium;
(b) applying the polymer solution to prepare at least two cell culture layers;
(c) placing one of the cells to be tested on the at least two cell culture layers;
(d) laminating the at least two cell culture layers so as to form a three-dimensional culture laminating layer having the one of the cells to be tested positioned in the three-dimensional culture laminating layer;
(e) preparing at least one cell-locating layer having a polyethylene glycol diacrylate (PEGDA) solution, the acoustic scattering medium solution, a plurality of photoacoustic markers and the cell culture medium; and
(f) positioning the at least one cell-locating layer in the three-dimensional culture laminating layer so as to form the three-dimensional cell culture system, wherein the at least one cell-locating layer has a preset shape and a preset position;
wherein the imaging system is constructed according to a theory selected from one of optics, acoustics, optoacoutics and acousto-optics, and the at least one cell image is one of an ultrasonic image, a photoacoustic image, an elasticity image, a molecular image and a distribution image.

13. The manufacturing method according to claim 12, wherein the monomer is formed by an acrylate and a polyethylene glycol (PEG), and the bio-molecule is one of an enzyme sensitive peptide (ESP), a growth factor and a chemokine.

14. The manufacturing method according to claim 13, wherein the enzyme sensitive peptide is formed by amino acids.

15. The manufacturing method according to claim 12, wherein the acoustic scattering medium solution is a SiO₂ suspension.

16. The manufacturing method according to claim 12, wherein the first polymerized solution has a percent weight in volume ranged from 3-5%, the second polymerized solution has a percent weight in volume ranged from 1-1.5%, the acoustic scattering medium solution has a percent weight in volume ranged from 0.1-0.3%, and the cell culture medium is formed by a Dulbecco's modified eagle medium, a fetal bovine serum (FBS) and antibiotics.

17. The manufacturing method according to claim 12, wherein the polyethylene glycol diacrylate solution of the cell-locating layer has a percent weight in volume ranged from 3-5%.

18. The manufacturing method according to claim 12, wherein the polymer solution and the cell-locating layer further include an ultraviolet-initiator having a percent weight in volume ranged from 0.1-1%.

19. The manufacturing method according to claim 12, wherein the photoacoustic marker is one of a nanogold rod, a dye and a graphite; wherein, in the case that the photoacoustic marker is the nanogold rod, a concentration of the nanogold rod in the cell-locating layer is larger than 5*10⁹ unit/ml.

20. The manufacturing method according to claim 12, wherein the cells to be tested are cells sampled or derived from one of a human organ and an animal and have a volume concentration ranged from 10⁴-10⁶ cell/ml.

21. The manufacturing method according to claim 12, wherein the preset shape of the cell-locating layer is a cylindrical shape.

22. The manufacturing method according to claim 12, wherein the imaging system is one of an ultrasonic system, an optoacoutic imaging system, an elastic imaging system and an optic imaging system.

23. A manufacturing method of a three-dimensional cell culture system, to produce the three-dimensional cell culture system for an imaging system to observe at least one cell image of cells to be tested, comprising the steps of:
(a) preparing a polymer solution having at least one monomer capable of photo-polymerization, a first polymerized solution formed by polymerizing bio-molecules capable of providing cell recognition and providing signals to one of the cells, a second polymerized solution formed by polymerizing an acrylate, a polyethylene glycol and a cell adhesive peptide (CAP), an acoustic scattering medium solution and a cell culture medium;
(b) applying the polymer solution to prepare at least two cell culture layers;
(c) laminating the at least two cell culture layers so as to form a three-dimensional culture laminating layer;
(d) preparing at least one cell-locating layer having a polyethylene glycol diacrylate (PEGDA) solution, the acoustic scattering medium solution, a plurality of photoacoustic markers and the cell culture medium;
(e) positioning the at least one cell-locating layer in the three-dimensional culture laminating layer so as to form the three-dimensional cell culture system, wherein the at least one cell-locating layer has a preset shape and a preset position; and
(f) placing one of the cells to be tested in the three-dimensional culture laminating layer of the three-dimensional cell culture system;
wherein the imaging system is constructed according to a theory selected from one of optics, acoustics, optoacoutics and acousto-optics, and the at least one cell image is one of an ultrasonic image, a photoacoustic image, an elasticity image, a molecular image and a distribution image.

24. The manufacturing method according to claim 23, wherein the monomer is formed by an acrylate and a polyethylene glycol (PEG), and the bio-molecule is one of an enzyme sensitive peptide (ESP), a growth factor and a chemokine.

25. The manufacturing method according to claim 24, wherein the enzyme sensitive peptide is formed by amino acids.

26. The manufacturing method according to claim 23, wherein the acoustic scattering medium solution is a SiO₂ suspension.

27. The manufacturing method according to claim 23, wherein the first polymerized solution has a percent weight in volume ranged from 3-5%, the second polymerized solution has a percent weight in volume ranged from 1-1.5%, the acoustic scattering medium solution has a percent weight in volume ranged from 0.1-0.3%, and the cell culture medium is formed by a Dulbecco's modified eagle medium, a fetal bovine serum (FBS) and antibiotics.

28. The manufacturing method according to claim 23, wherein the polyethylene glycol diacrylate solution of the cell-locating layer has a percent weight in volume ranged from 3-5%.

29. The manufacturing method according to claim 23, wherein the polymer solution and the cell-locating layer further include an ultraviolet-initiator having a percent weight in volume ranged from 0.1-1%.

30. The manufacturing method according to claim 23, wherein the photoacoustic marker is one of a nanogold rod, a dye and a graphite; wherein, in the case that the photoacoustic marker is the nanogold rod, a concentration of the nanogold rod in the cell-locating layer is larger than 5*10⁹ unit/ml.

31. The manufacturing method according to claim 23, wherein the cells to be tested are cells sampled or derived from one of a human organ and an animal and have a volume concentration ranged from 10⁴-10⁶ cell/ml.

32. The manufacturing method according to claim 23, wherein the preset shape of the cell-locating layer is a cylindrical shape.

33. The manufacturing method according to claim 23, wherein the imaging system is one of an ultrasonic system, an optoacoutic imaging system, an elastic imaging system and an optic imaging system.
